(19) United States
(12) Patent Application Publication
(10) Pub. No.: US 2018/0360751 A1
(43) Pub. Date: Dec. 20, 2018

(54) Transdermal cream for treating Dupuytren's Contracture

(71) Applicant: Advanced Fibrosis Research LLC, Sheridan, WY (US)

(72) Inventors: Latha Satish, Blue Ash, OH (US); Carl Reese, Poway, CA (US); Sandeep Kathju, Sewickley, PA (US); Mark Baratz, Bethel Park, PA (US)

(73) Assignee: Advanced Fibrosis Research LLC, Sheridan, WY (US)

(21) Appl. No.: 16/009,527

(22) Filed: Jun. 15, 2018

Related U.S. Application Data
(60) Provisional application No. 62/521,593, filed on Jun. 19, 2017.

(51) Int. Cl. A61K 9/06; A61K 9/00; A61P 21/00; A61P 19/00; A61K 31/4412; A61K 47/24

(52) U.S. Cl. CPC A61K 9/06; A61K 9/0014; A61K 47/24; A61P 19/00; A61K 31/4412; A61P 21/00

(57) ABSTRACT

A pharmaceutically acceptable transdermal composition for the treatment of patients with musculoskeletal connective tissue fibrosis or a disorder including Dupuytren's Contracture, Peyronie's disease, Ledderhose disease, or Knuckle Pads, includes an active composition and a base composition. The active composition is present in the transdermal composition in an amount of about 10% to about 40% by weight of the transdermal composition and includes one or more growth factor antagonists. The base composition is present in an amount of about 60% to about 90% by weight of the transdermal composition and comprises a transdermal delivery system or transdermal carrier. A method of treating patients with musculoskeletal connective tissue fibrosis, including topically applying the transdermal composition in effective amount to the affected tissue is further disclosed.

20

### FIELD OF THE INVENTION

The present invention relates to a pharmaceutically acceptable transdermal composition for the treatment of patients with musculoskeletal connective tissue fibrosis. The invention also relates to a method of treating patients with musculoskeletal connective tissue fibrosis.

BACKGROUND INFORMATION

Fibrotic disorders of musculoskeletal connective tissues are progressive diseases that affect the functioning of appendageal organs. They are characterized by excessive accumulation of connective tissue resulting in slow but continuous tissue contraction. The consequence is progressive deterioration in the normal structure and function of affected organs. Musculoskeletal fibro-proliferative connective tissue diseases include Dupuytren's Contracture, Ledderhose Disease, Peyronie's Disease, and Knuckle Pads. In particular, Dupuytren's Contracture (DC) leads to irreversible flexion of one or more fingers.

Dupuytren's Contracture alone affects more than 7% of the US population. Both men and women are affected although men more often than women. The diseases typically appear as one ages. They are commonly characterized as “wound repair run amuck” because they resemble impaired healing.

In recent years, research in diverse fields (e.g., Idiopathic Pulmonary Fibrosis (IPF) research) has increasingly highlighted the role of wound-repair growth factors, especially Transforming Growth Factor Beta 1 (TGFβ1), as primary contributors to the molecular mechanism of fibrosis.

Transforming growth factor-β1 (TGF-β1) and other growth factors exist in excessive concentrations in diseased tissues, especially near the capillary beds that infuse the diseased tissues.

These growth factors cause proliferation of fibroblasts, which in turn, transform into myofibroblasts. Myofibroblasts are the primary cells responsible for the disabling tissue contraction that is the hallmark of fibrosis. The growth factors also stimulate production of collagenase fibers in the extracellular matrix (ECM). They do this by directly interacting with the ECM through fibronectin and indirectly by stimulating myofibroblasts to produce collagenase fibers. And finally, the growth factors stimulate contraction of the collagenase fibers by directly interacting with the ECM and through the myofibroblasts.

Certain classes of anti-fibrotic agents have been shown to work in other fibrotic diseases by inhibiting the production and activity of TGFβ1 and other growth factors. Table 1 below shows the deleterious activities of fibrosis.

TABLE 1
Deleterious Fibrosis Activities

1 Excessive Expression of Wound-Repair Growth Factors

a) Transforming Growth Factor Beta 1 (TGFβ1)
b) Platelet-Derived Growth Factor (PDGF)
c) Vascular Endothelial Growth Factor (VEGF)
d) Basic Fibroblast Growth Factor (bFGF)

2 Excessive Fibroblast Activity

a) Proliferation
b) Migration
c) Transformation into Myofibroblasts
d) Contraction

3 Excessive Production of Extracellular Matrix (ECM) Proteins

a) Collagen
b) Fibronectin

The current and most common treatment strategies include surgery, collagenase, percutaneous needle fasciectomy, and steroidal injections. The first three of these treatments involve the process of removing, debriding, or cutting the tissue. This is a form of controlled injury that further stimulates wound repair, which in turn runs a high risk of turning fibrotic. Furthermore, these three treatments are only provided once a patient's disease has progressed to the point of debilitation. For example, patients with Dupuytren's Contracture typically wait until contractures reach 60 degrees or more before surgery is arranged. In other words, patients are told, “Wait until it gets worse—much worse.” This is standard protocol because recurrence is high, and the costs for these treatments are high. None of these procedures are especially effective, and all are accompanied by high recurrence rates.

Pharmacological therapies are uncommon or not yet approved. Steroidal injections may offer temporary relief, but they must be administered judiciously. Repeated injections into the diseased tissue, which is usually necessary, may cause surrounding tissues to break down.

There remains a need for an effective treatment of fibrotic disorders of musculoskeletal connective tissues that can be administered during the early stages, advanced stages, or as part of a rehabilitation program after invasive procedures to prevent recurrence. A noninvasive treatment is preferred. This treatment will slow, halt, and even reverse the disease.

For example, pirfenidone is a commercially available oral therapy for the treatment of IPF. Pirfenidone safely slows or arrests enlargement of fibrotic lesions and prevents new lesions after injury. At a molecular level, it is understood that pirfenidone is an inhibitor of the wound-repair growth factors (see Table 1). The ability of pirfenidone to combat the fibrosis seen in musculoskeletal connective tissue fibrotic diseases (e.g., Dupuytren's Contracture) has not been previously reported.

### SUMMARY OF THE INVENTION

The present invention is directed to a pharmaceutically acceptable transdermal composition for the treatment of patients with musculoskeletal connective tissue fibrosis or a disorder including Dupuytren's Contracture, Peyronie's disease, Ledderhose disease, or Knuckle Pads. More particularly, the transdermal composition of the present invention includes an active composition and a base composition. The active composition is present in the transdermal composition in an amount of about 10% to about 40% by weight of the transdermal composition and includes one or more growth factor antagonists, that antagonize one or more growth factors such as Transforming Growth Factor Beta 1 (TGFβ1), Platelet-Derived Growth Factor (PDGF), Vascular Endothelial Growth Factor (VEGF), and Basic Fibroblast Growth Factor (bFGF or FGF2). The base composition is present in an amount of about 60% to about 90% by weight of the transdermal composition and comprises a transdermal delivery system or transdermal carrier. The active composition may include pirfenidone as an active ingredient and the base composition may include a phospholipid base as a transdermal delivery system, e.g., Lipoderm®.

The composition of the present invention may also include wetting agents, buffering agents, diluting agents, stabilizing agents, emulsifiers, dispersing agents, preservatives, antioxidants, and/or mixtures thereof.

The present invention is also directed to a method of treating patients with musculoskeletal connective tissue fibrosis, for example, the method including topically applying a transdermal composition, which includes an active composition and a base composition, such as those described above, in effective amount to the affected tissue. Preferably, the transdermal composition is applied to a predetermined area of the skin to deliver a therapeutically effective amount of the active agent to a patient.

For purposes of the present invention, a transdermal composition includes a transdermal delivery system or component or carrier or vehicle, which allows delivery of the active agent through the skin of a patient. Further, topical application includes application to a predetermined area, preferably unbroken, of the skin of a formulation in the form of a cream, gel, ointment, or paste.

The present invention is further directed to a method of preparing a transdermal composition, which includes an active composition and a base composition, such as described above.

### DETAILED DESCRIPTION OF THE INVENTION

For purposes of the following detailed description, it is to be understood that the invention may assume various alternative variations and step sequences, except where expressly specified to the contrary. Moreover, other than in any operating examples, or where otherwise indicated, all numbers expressing, for example, quantities of ingredients used in the specification and claims are to be understood as being modified in all instances by the term “about”. Accordingly, unless indicated to the contrary, the numerical parameters set forth in the following specification and attached claims are approximations that may vary depending upon the desired properties to be obtained by the present invention. At the very least, and not as an attempt to limit the application of the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard variation found in their respective testing measurements.

Also, it should be understood that any numerical range recited herein is intended to include all sub-ranges subsumed therein. For example, a range of “1 to 10” is intended to include all sub-ranges between (and including) the recited minimum value of 1 and the recited maximum value of 10, that is, having a minimum value equal to or greater than 1 and a maximum value of equal to or less than 10.

In this application, the use of the singular includes the plural and plural encompasses singular, unless specifically stated otherwise. In addition, in this application, the use of “or” means “and/or” unless specifically stated otherwise, even though “and/or” may be explicitly used in certain instances. Further, in this application, the use of “a” or “an” means “at least one” unless specifically stated otherwise.

Transdermal delivery of antifibrotic agents offers the following distinct advantages over current treatment strategies. It targets wound-repair growth factors, i.e., targets primary instigators of dysregulated wound repair. It provides an early treatment option by treating diseased tissue before contractions occur. It offers targeted therapy and lower the risk of side effects. It is noninvasive and, thus, avoids high risks and recovery times of invasive treatments. It offers a viable post-surgery therapy by reducing the risk of recurrence. These advantages are further detailed below.

Wound Repair Growth Factors

Using growth factor antagonists, which antagonize one or more growth factors selected from the group consisting of Transforming Growth Factor Beta 1 (TGFβ1), Platelet-Derived Growth Factor (PDGF), Vascular Endothelial Growth Factor (VEGF), and Basic Fibroblast Growth Factor (bFGF or FGF2), present an attractive therapy by targeting the growth factors most associated with fibrosis. Moreover, a subclass of these anti-fibrotic agents, of which pirfenidone is one member, are small lipophilic molecules without polarity (nonpolar) that are ideal candidates for transdermal delivery.

Early Treatment

Transdermal delivery of anti-fibrotic agents offers the opportunity to treat the disease at a much earlier stage and hopefully prevent contractions from ever occurring.

Targeted Therapy

Oral medications have the disadvantage of potentially impacting organs not meant for targeting. They also suffer from reduced bioavailability as a result of first-pass metabolism. Transdermal delivery on the other hand provides a more localized delivery of the anti-fibrotic agent to the diseased tissue.

Noninvasive

Transdermal delivery of anti-fibrotic agents is noninvasive. Invasive techniques incur the risks of infections. Moreover, invasive techniques such as surgery are traumatic to the affected organs. Surgery is a form of tissue injury, which stimulates the wound repair response, which raises the levels of wound-repair growth factors and the risk of recurrence.

Viable Post-Surgery Therapy

Post-treatment therapy of invasive methods usually involves physical therapy. However, it appears that physical therapy does little to prevent recurrence. Application of transdermal cream as part of post-surgery therapy may provide a way to prevent recurrences.

In this context, the present invention provides a transdermal composition comprising, consisting essentially of, or consisting of, an active composition and a base composition. The transdermal composition comprises, consists essentially of, or consists of, an amount of about 10% to about 40% by weight of active composition and comprises, consists essentially of, or consists of, base composition. The base composition comprises, consists essentially of, or consists of, a transdermal delivery system or transdermal carrier. The transdermal delivery system or transdermal carrier comprises, consists essentially of, or consists of, a phospholipid base such as Lipoderm®. The active composition comprises, consists essentially of, or consists of, pirfenidone.

Pirfenidone is also known as 5-methyl-l-phenylpyridin-2-one. Transdermal creams may include lecithin organogels. Lecithin organogels are a class of vehicles for the delivery of bioactive agents through the skin and may include a variety of components such as, for example, lecithin, polymers (polyethylene glycol PEG), natural extracts, alcohols, water, glycerin, oils such as shea butter and coconut oil, ascorbyl palmitate, xanthan gum, and disodium EDTA. However, phospholipid bases such as Lipoderm®, which include liposomal components have been shown to have superior qualities as transdermal delivery vehicles.

The present invention is also directed to a method of treating patients with musculoskeletal connective tissue fibrosis, for example, the method comprising topically applying a transdermal composition, which comprises the active composition and the base composition described above, in effective amount to the affected tissue.

The present invention is further directed to a method of preparing a transdermal composition, which comprises an active composition and a base composition, such as described above.

Any of the transdermal compositions described herein can include additional ingredients or additives. Non-limiting examples of additional ingredients or additives that can be used with the transdermal compositions of the present invention include wetting agents, buffering agents, diluting agents, stabilizing agents, emulsifiers, dispersing agents, preservatives, antioxidants, solvents, and/or mixtures thereof

The following examples are presented to demonstrate the general principles of the invention. The invention should not be considered as limited to the specific examples presented. All parts and percentages in the examples are by weight unless otherwise indicated.

### EXAMPLE 1

Transdermal compositions of varying strengths according to the present invention may be prepared or manufactured by the following method. A transdermal composition that contains 10% of an active agent is manufactured by following the steps described below.
Step 1. In the powder hood, pirfendione is weighed and triturated to a fine powder with a mortar and pestle.
Step 2. This process is done until a fine powder is formed. Following which, slowly added approximately 10% of the final volume of Lipoderm and hand mixed with trituration in mortar and pestle.
Step 3. The mixture from the above is backloaded to an appropriate size syringe and the volume of the mixture is measured.
Step 4. In a second syringe, backloaded enough Lipoderm cream to reach the final volume needed. (For example, if 5 mL is measured in step 3 and the total volume needed is 10 mL and then 5 mL of Lipoderm is added to reach the final needed volume.
Step 5. Syringes from steps 3 and 4 are attached using a Luer-Lock-to-Luer Lock connector. The 2 syringes are mixed back and forth at least forty times or until a uniform mixture is formed.
Step 6. Using the Luer Lock to oral syringe adaptor, dispensed in Amber 1 mL oral syringes and labeled as external use only.

One hundred (100) grams of 10% transdermal cream is manufactured by selecting 10% by weight of active ingredient (10 grams) and 90% by weight of base (90 grams). A cream of greater strength, say 20%, is manufactured by using 20% by weight active ingredient and 80% by weight base and repeating steps (1) and (2) above. Maximum strength is 40% by weight of active ingredient.

In an aspect of the invention, transdermal compositions of varying strength are manufactured by following the same method. A transdermal cream of 10% pirfenidone is manufactured by
1) Mixing 10% by weight of pirfenidone with 90% by weight of Lipoderm®.
2) Agitate until the mix is homogenous.

### EXAMPLE 2

A cream of greater strength, say 20% pirfenidone, is manufactured by using 20% by weight pirfenidone and 80% by weight Lipoderm® and repeating steps (1) and (2) above. Maximum strength is 40% by weight of pirfenidone.

### EXAMPLE 3

Example of Use

The transdermal cream is intended to be used during one or more of the following stages:
1) Early stage;
2) Advanced stage;
3) Post-surgery or post-collagenase therapy;

Administration is similar for all three stages, although dosages may vary. The cream must be applied consistently to ensure the most efficacious outcome.
The initial application is a 10% by weight active ingredient. It is applied to the affected areas twice per day, approximately twelve hours apart. The number of applications is increased to three after one week and four after two weeks. Administer the applications evenly across the day as much as possible. Three applications should be given 8 hours apart and four applications should be given six hours apart.
Improvements occur over time. Maintain the regimen for four weeks. Since objective assessments on the part of patients can be misleading, it is important to maintain appointments with doctors who should keep notes to compare conditions from exam to exam. Increases in dosage from 10% to a maximum of 30% may be needed.
Continue the regimen until the desired outcome is achieved.

### Early Stage

The most effective management is early recognition and treatment before the development of tissue contracture. A patient may visit a doctor complaining of a “bump” on the affected organ. He or she may describe it as “hard” and somewhat “painful” when compressed. This is the fibrous mass or nodule that is the first visible or palpable sign of fibroplasia. Application of the cream at this stage may arrest the cascade of growth factors, fibroblasts, and collagen synthesis. The cream should be applied up to the maximum dose and maintained at the maximum dose until the fibrous mass softens, shrinks, and disappears.

### Advanced Stage

During the middle stages, tissue contraction has begun. Once again application of the cream at this stage may slow or even halt progression of the disease. Application of the cream may be required on an ongoing basis to prevent further progression.

### Post-Surgery or Post-Collagenase

Application of the cream to the affected areas after surgery or after treatment with collagenase may reduce or eliminate the chances of recurrences. Recurrence rates are as high as 80% depending on the study quoted. One reason is that these invasive techniques are traumatic and initiate a significant wound-repair response. This results in the entire area being flooded with growth factors, inflammatory cells, and other wound-repair factors. The cream should be applied around the area of the surgery but not on the sutures within three of four days of surgery. Titrate to the maximum dosage and then maintain that dosage until the area heals entirely. Note this may be several months to as long as one year.

### Precautions

Applications should be stopped if the skin develops a rash. Titrate again to the maximum tolerable dose once the rash disappears.

### EXAMPLE 4

Studies on the transdermal delivery to rats' forepaw of a compositions according to the present invention are described below.

### Brief Description of the Protocol

Total no. of animals used—8 male and female athymic (nude) rats
4 animals received 15% topical application of pirfenidone composition (15% PFD)
4 animals received 30% topical application of pirfenidone composition (30% PFD)
One animal placed in the 30% topical application group died on the day when the treatment started (pathological results showed that animal was delivered from the vendor with some genetic disorder).
15 and 30 mg of pirfenidone in 100 μl Lipoderm based cream was topically rubbed into the forepaw of the rats once every day for a period of 29 days. Blood samples were collected on days 0, 7, 14, 21, and on day 29, times of collection were at 0, 4, 8, 12, and 24 hours. Animals were sacrificed on day 30 and various tissues from the forepaws were harvested.
Tissues collected were the following: skin, palmar fascia, muscle, and tendon.
The amount of drug in the blood and tissues were determined via mass spectrometry analyses.
The results of these studies are presented in the following tables.

TABLE 2
Amount of Pirfenidone Topically Applied to Rats Forepaw

| Days | 15% PFD | 30% PFD |
|---|---|---|
| Day 7 | 105000 μg | 210000 μg |
| Day 14 | 210000 μg | 420000 μg |
| Day 21 | 315000 μg | 630000 μg |
| Day 29 0 h | 420000 μg | 840000 μg |
| Day 29 4 h | 420000 μg | 840000 μg |
| Day 29 8 h | 420000 μg | 840000 μg |
| Day 29 12 h | 420000 μg | 840000 μg |
| Day 29 24 h | 420000 μg | 840000 μg |

The protocol for plasma separation from blood for mass spectrometry analysis was as follows.

Tail vein blood collection was done on specified days. Blood was collected into heparinized tubes coated with 33 IU of heparin. Blood collected was around 300 μl. Blood samples were mixed well and spun down at 1,500×g for 10 min at room temperature. Plasma was separated and stored at −80° C. until use. Plasma was subjected to Mass Spectrometry analysis.

The mass spectrometry determination of pirfenidone levels in rat plasma at different days of collection for transdermal compositions containing 15% and 30% by weight of pirfenidone shows an initial burst of drug release in the blood but it was significantly low compared to the amount of drug that was topically applied. After 29 days, the amount of pirfenidone detected in the plasma was very low. 6 animals were used for this experiment. 3 animals received 15% PFD and 3 animals received 30% PFD.

The protocol for drug extraction from tissues was as follows.
Frozen tissue samples were weighed and broken down using hammer and pulverized and homogenized with 1 ml of methanol (100%) using vortex (2×5 mins) with mid-high speed for cell disruption at cold room.
The homogenate was subjected to sonication in the cold room for 6 hours and then left overnight at room temperature to allow for the complete extraction of drug from the tissue.
The homogenate was centrifuged at 2100 rpm for 10 min. The supernatant (methanol) was transferred to a microcentrifuge tube and left overnight in the hood to evaporate.
The drug residue was reconstituted with 500 μl of rat plasma and dissolution using a spinner for 1 hour at room temperature. Drug in the plasma was analyzed and tissue drug concentration was expressed in ng/g of tissue weight.

TABLE 3
Mass Spectrometry Determination of Pirfenidone
Levels in Rat Tissues (15% PFD)

| | PFD in ng/mg | Tissue weight | Total amount of |
|---|---|---|---|
| 15% PFD ¹ | of tissue | in mgs. | PFD in ngs |
| R-Skin ² | 2.525 | 33.25 | 83.95625 |
| R-PF ³ | 4.65 | 7.5 | 34.875 |
| R-Tendon | 0.475 | 14.66667 | 6.96666825 |
| R-Muscle | 2.053333333 | 10.66667 | 21.90222907 |
| L-skin ⁴ | 3.766666667 | 35.3333 | 133.0887633 |
| L-PF ³ | 1.06 | 11.66667 | 12.3666702 |
| L-Tendon | 2.696666667 | 14.66667 | 39.5511201 |
| L-muscle | 2.126666667 | 12.66667 | 26.93778487 |

¹15% PFD - Even with small amount of palmar fascial tissue there was considerable amount of drug determined in the right forepaw that received Pirfenidone. Though only the right fore paw received the drug we see significant amount of drug in the left forepaw tissues as well. This might be due to the fact that there was cross-contamination between the paws which was difficult to control. Efforts were made to rub the cream longer onto the right forepaw to ensure that the entire cream was absorbed.
² R = Right Forepaw
³ PF = Palmar Fascia
⁴ L = Left Forepaw

TABLE 4
Mass Spectrometry Determination of Pirfenidone
Levels in Rat Tissues (30% PFD)

| | PFD in ng/mg | Tissue weight | Total amount of |
|---|---|---|---|
| 30% PFD ¹ | of tissue | in mgs. | PFD in ngs |
| R-skin | 3.77 | 46.333333 | 174.6766654 |
| R-PF | 14.05666667 | 5.333333 | 74.9688842 |
| R-Tendon | 4.933333333 | 24.66666666 | 121.6888889 |
| R-muscle | 12.93666667 | 8.333333 | 107.8055512 |
| L-skin | 6.33 | 47.66666666 | 301.73 |
| L-PF | 4.306666667 | 12.333333 | 53.11555412 |
| L-Tendon | 7.773333333 | 12.66666666 | 98.46222217 |
| L-muscle | 4.396666667 | 12.666667 | 55.69111258 |

¹ Increased amount of drug was observed with higher concentration of PFD.

### EXAMPLE 5

Stability of pirfenidone (15% PFD) in Lipoderm at room temperature was also determined via HPLC after 30, 60, and 90 days. These measurements are provided in Table 5 below and show that the pirfenidone composition (15% PFD) does not lose its potency after 30, 60, and even 90 days.

TABLE 5
HPLC Determination of the Potency of Pirfenidone
Lipoderm Composition 15% PFD at Room Temperature
(20 to 25° C.) at 30, 60, and 90 Days

| | Expected | Measured | Measured vs. |
|---|---|---|---|
| Day | Amount | Amount | Expected Ratio |
| 30 | 15% | 15.31 | 102.1% |
| 60 | 15% | 16.18 | 107.8% |
| 90 | 15% | 16.42 | 109.4% |

Whereas particular embodiments of this invention have been described above for purposes of illustration, it will be evident to those skilled in the art that numerous variations of the details of the present invention may be made without departing from the invention as defined in the appended claims.

We claim:

1. A transdermal composition for the treatment of patients with musculoskeletal connective tissue fibrosis, the transdermal composition comprising an active composition and a base composition.

2. The transdermal composition of claim 1, wherein the active composition comprises pirfenidone.

3. The transdermal composition of claim 1, wherein the base composition comprises a phospholipid base.

4. The transdermal composition of claim 1, wherein the transdermal composition comprises about 10% to about 40% by weight of the active composition.

5. The transdermal composition of claim 1, wherein the transdermal composition comprises 60% to 90% by weight of the base composition.

6. A transdermal composition for the treatment of patients with a musculoskeletal connective tissue disorder selected from the group consisting of Dupuytren's Contrature, Peyronie's Disease, Ledderhose Disease, and Knuckle Pads, the transdermal composition comprising:
a) an active composition present in an amount of about 10% to 40% by weight of the transdermal composition, the active composition comprising one or more growth factor antagonists, the antagonists antagonize one or more growth factors selected from the group consisting of Transforming Growth Factor Beta 1 (TGFβ1), Platelet-Derived Growth Factor (PDGF), Vascular Endothelial Growth Factor (VEGF), and Basic Fibroblast Growth Factor (bFGF or FGF2);
b) a base composition present in an amount of about 60% to 90% by weight of the transdermal composition, the base composition comprising a transdermal cream.

7. The transdermal composition of claim 6, wherein the active composition comprises pirfenidone.

8. The transdermal composition of claim 6, wherein the base composition comprises a phospholipid base.

9. A transdermal composition for the treatment of patients with Dupuytren's Contracture (DC) comprising:
a) an active composition present in an amount of about 10% to 40% by weight of said transdermal composition, the active composition comprising pirfenidone;
b) a base composition present in an amount of about 60% to 90% by weight of said transdermal composition, the base composition comprising a phospholipid base, wherein the base composition delivers the active composition directly to the diseased tissue of the patients with DC.

10. A method of treating patients with musculoskeletal connective tissue fibrosis, the method including topically applying the transdermal composition of claim 1 in effective amount to an affected tissue.

11. A method of preparing the transdermal composition of claim 1 comprising:
a) mixing about 10% to about 40% by weight of active composition with about 60% to about 90% by weight of base composition; and
b) agitate the mix until a homogenous composition is obtained.
